(19) United States
(12) Patent Application Publication
(10) Pub. No.: US 2025/0280850 A1
(43) Pub. Date: Sep. 11, 2025

(54) WHOLE GRAIN SYRUPS AND FLOURS

(71) Applicant: Paragon Flavors, Inc., Princeton, NJ (US)

(72) Inventors: Matthew SILLICK, Montgomery, NJ (US); Christopher Mark GREGSON, Princeton, NJ (US)

(73) Assignee: Paragon Flavors, Inc., Princeton, NJ (US)

(21) Appl. No.: 19/186,685

(22) Filed: Apr. 23, 2025

Related U.S. Application Data
(63) Continuation of application No. 16/991,330, filed on Aug. 12, 2020, now abandoned.
(60) Provisional application No. 62/885,752, filed on Aug. 12, 2019.

(51) Int. Cl. A23G 3/34; A23L 7/10; A23L 7/104; A23L 33/24; A23L 33/29

(52) U.S. Cl. CPC A23G 3/343; A23L 7/101; A23L 7/104; A23L 7/197; A23L 33/24; A23L 33/29; C12Y 302/01001

(57) ABSTRACT

A low sugar, low viscosity syrup from whole grain seed or flour is disclosed. A readily soluble flour is also disclosed. The syrup comprises 90% to 100% of its solid components as water soluble solids and the wholegrain flour comprises 50% to 95% of its solid components as water soluble solids. By controlling the mashing and hydrolysis of a cooked whole grain seed or flour, a syrup can be obtained comprising a defined oligosaccharide content with a narrow molecular weight distribution (i.e. low in sugar and low DP 11+).

### CROSS-REFERENCE TO RELATED APPLICATIONS

This application is a U.S. Continuation application that claims priority to a U.S. Non-Provisional patent application Ser. No. 16/991,330 filed on Aug. 12, 2020 which further claims priority to U.S. Provisional Patent Application No. 62/885,752 filed on Aug. 12, 2019, the contents of which are hereby incorporated by reference in their entireties.

### FIELD OF THE EMBODIMENTS

The field of the invention and its embodiments relate to whole grain syrups and flours. In particular, the invention and its embodiments relate to low sugar, low viscosity whole grain syrups and readily-soluble sprouted wholegrain flours.

### BACKGROUND OF THE EMBODIMENTS

Exogenous enzymes (exo-enzymes) may be added to food products to promote a desirable effect. Endogenous enzymes naturally exist in food and may affect the quality of the foods (either positively or negatively) by their actions. One of the most cognitive endogenous enzymes is pectin methylesterase (PME), which is found in plants and bacteria. Multiple forms of PME (e.g., basic, neutral, and acidic isoforms) can be present within each species. The control of PME activity has been a common subject of study because of the implications in the modification of the texture of fruit and vegetables and as a destabilizing agent of pectin materials in fruit juices and concentrates.

As another example, malt extracts use endogenous enzymes and start with whole grain, but are exposed to lautering, which removes most of their protein, lipids, and fiber, rendering the nutritional profile of the extract different from that of the flour. Sprouted flours or sweet flours are known, but they are not readily soluble, and, therefore, unsuitable for replacing highly refined and soluble ingredients, such as maltodextrin. As such, there is an ongoing need for a readily soluble wholegrain flour that retains the nutritional profile of the original grain.

Syrups are solutions of saccharides widely used in foods, for example, to impart properties such as sweetness or bulking. Natural enzymes, such as those within koji (Aspergillus oryzae mold), may be used with whole grains, such as rice, to produce syrup. Traditionally, syrups can be made by processing sweet juices, such as cane juice, sorghum juice, maple sap, or agave nectar. More recently, the large-scale production of syrups for the food industry relies on the enzymatic or acid hydrolysis of refined starches. For example, corn starch can be converted into corn syrup or high fructose corn syrup, a common additive of various beverages and processed foods. Such refined syrups lack many of the nutrients that were originally present in the whole grain.

The potential applications of syrups in foods is limited because of their viscosity, high sweetness, and/or lack of nutrients. There is therefore an ongoing need for improved syrup compositions that contain minimal sweetness, minimal viscosity, and a nutrient composition close to that of the whole grain.

Examples of related art are described below:

U.S. Pat. No. 4,756,912 relates to whole grain rice, either white or brown rice, that is liquefied and treated with high levels of a glucosidase enzyme and/or a combination with betaamylase enzyme in a saccharification step. Total enzymatic reaction time is limited to about four hours for both the liquefaction and saccharification steps combined to prevent the development of undesirable off-flavors. The product of the saccharification step is partially clarified to remove substantially all rice fiber, but not other nutritional components and then concentrated to produce a preferred rice syrup sweetener which is cloudy in character and has a solids composition defined as follows: soluble complex carbohydrates (about 10 to 70% of solids), maltose (about 0 to 70% of solids), glucose (about 5 to 70% of solids), ash or minerals (about 0.1 to 0.6% of solids), and protein and fat (about 1 to 3.5% of solids). The rice syrup sweetener can be dried.

WO 2012/078150 relates to syrups comprising a content of sweetening agent above 15% by weight of the syrup, a hydrolyzed whole grain composition and, an alpha-amylase or fragment thereof, which alpha-amylase or fragment thereof shows no hydrolytic activity towards dietary fibers when in the active state, and wherein the syrup has a water activity above 0.6.

Various whole grain syrups and flours exist in the art. However, these whole grain syrups and flours are substantially different from the present disclosure, as the other inventions fail to solve all the problems taught by the present disclosure.

### SUMMARY OF THE EMBODIMENTS

The present invention and its embodiments relate to whole grain syrups and flours. In particular, the invention and its embodiments relate to low sugar, low viscosity whole grain syrups and readily-soluble sprouted wholegrain flours.

A first embodiment of the present invention describes a syrup formed from a processed seed. The syrup has a composition comprising: less than 25% of monosaccharides and disaccharides in a dry weight basis, greater than 55% of oligosaccharides in the dry weight basis, less than 20% of polysaccharides in the dry weight basis, and non-starch derived nutritional components from a grain present in a range of approximately 1% to approximately 15%. The syrup comprises 90% to 100% of its solid components as water soluble solids.

Further, the monosaccharides and disaccharides comprise a sweetness index below approximately 0.15. The polysaccharides have a degree of polymerization of at least 11. The non-starch derived nutritional components include proteins, fats, minerals, cellulose, and soluble fibers. Further, the processed seed does not contain a refined starch and has not been fermented by a microorganism.

The syrup may be derived from a sprouted wholegrain source without use of exogenous enzymes. No exogenious amylase is added to the sprouted wholegrain source. The syrup is used as a food binder, an encapsulating agent, a humectant, a mouthfeel enhancer, a dispersant, a solvent, a carrier, an adhesive, and/or a metabolic energy source.

A second embodiment of the present invention describes a method for producing a syrup. The method includes: mashing a mixture comprising a cooked whole grain cereal in a presence of an added enzyme at a temperature of approximately 70° C. such that the added endoglycosidase converts starches of the mixture into oligosaccharides; homogenizing the mashed mixture; and filtering the homogenate to produce the syrup. The syrup has a Brix value in the range of about 10 to about 50. The syrup comprises less than about 20% polysaccharides on a dry weight basis with a degree of polymerization of at least about 11 (DP 11+).

The method also includes concentrating the homogenate to a Brix value of at least about 60 and/or deploying one or more digestive enzymes to break down cell wall structures, enhance syrup extraction, and increase levels of soluble fibers. The one or more digestive enzymes may include a peptidase, a pectinase, a pullanase, a proteinase, and/or a cellulase.

A third method of the present invention describes a method to create a wholegrain flour. The method includes: mashing a sprouted grain to activate endogenous enzymes of the sprouted grain and partially solubilize proteins and starches of the sprouted grains;

mechanically treating the mashed sprouted grain to reduce a D(4,3) average particle size of remaining insoluble constituents below 100 microns such that the insoluble constituents can be more readily accommodated within food applications; and drying the mashed sprouted grain to create a wholegrain flour powder or granule. The wholegrain flour is used as a bulking agent for a food product, a binder for the food product, a texturizer for the food product, a mouthfeel enhancer for the food product, a moisture control agent for the food product, a powder carrier for the food product, a dust-on application for the food product, and/or an encapsulating agent.

In examples, the sprouted grain is rice. In other examples, the sprouted grain is approximately 50% to approximately 95% soluble. In further examples, the sprouted grain is not an extract and maintains the nutritional profile of the whole grain. Moreover, the wholegrain flour has been spray dried. In these examples, the spray dried wholegrain flour has at least two times as much specific surface area as measured by gas absorption compared to a fine dry milled flour of a same grain and with the same average particle size. The method may further include filtering the mashed sprouted grain to remove coarse insoluble particles without substantially altering a nutritional profile of the mashed sprouted grain.

In general, the present invention succeeds in conferring the following benefits and objectives.

It is an objective of the present invention to provide a whole grain syrup with minimal sweetness and with minimal viscosity.

It is an object of the present invention to provide a sprouted wholegrain flour that retains its original nutritional profile of micronutrients and macronutrients and is suitable for replacing highly refined ingredients, such as maltodextrin, within prepared foods.

It is an object of the present invention to provide a wholegrain flour powder that can serve as a plating medium for oily food ingredients such as botanical extracts, flavor compositions, nutritional oils, and nut pastes.

It is an object of the present invention to provide a wholegrain flour powder useful for improving spice blend powders and/or for dust-on applications to food products, such as snack chips.

### BRIEF DESCRIPTION OF THE DRAWINGS

FIG. 1 depicts conversion tables between a refractive index and the Brix value at 20° C. and between the density and the Brix value at 20° C., according to at least some embodiments disclosed herein.

FIG. 2 depicts a block diagram of a method for producing a wholegrain syrup, according to at least some embodiments disclosed herein.

FIG. 3 depicts a block diagram of a method for producing a wholegrain flour, according to at least some embodiments disclosed herein.

FIG. 4 depicts a tabular representation of a typical nutritional profile range for brown rice, milled rice, and rice bran, according to at least some embodiments disclosed herein.

FIG. 5 depicts black pepper in a shape of a centrifuge tube plug of Example 6, according to at least some embodiments disclosed herein.

FIG. 6 depicts a mixture of black pepper and a brown rice flour mixture of Example 6 according to at least some embodiments disclosed herein.

### DESCRIPTION OF THE PREFERRED EMBODIMENTS

The preferred embodiments of the present invention will now be described with reference to the drawing. Identical elements in the various figures are identified with the same reference numerals.

Reference will now be made in detail to each embodiment of the present invention. Such embodiments are provided by way of explanation of the present invention, which is not intended to be limited thereto. In fact, those of ordinary skill in the art may appreciate upon reading the present specification and viewing the present drawing that various modifications and variations can be made thereto.

The phrase “and/or,” as used herein in the specification and in the claims, should be understood to mean “either or both” of the elements so conjoined, i.e., elements that are conjunctively present in some cases and disjunctively present in other cases. Thus, as a nonlimiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

When the term “about” is used in conjunction with a numerical range, it modifies that range by extending the boundaries above and below those numerical values. In general, the term “about” is used herein to modify a numerical value above and below the stated value by a variance of 20%, 10%, 5%, or 1%. In certain embodiments, the term “about” is used to modify a numerical value above and below the stated value by a variance of 10%. In certain embodiments, the term “about” is used to modify a numerical value above and below the stated value by a variance of 5%. In certain embodiments, the term “about” is used to modify a numerical value above and below the stated value by a variance of 1%.

When a range of values is listed herein, it is intended to encompass each value and subrange within that range. For example, “1-5 ng” is intended to encompass 1 ng, 2 ng, 3 ng, 4 ng, 5 ng, 1-2 ng, 1-3 ng, 1-4 ng, 1-5 ng, 2-3 ng, 2-4 ng, 2-5 ng, 3-4 ng, 3-5 ng, and 4-5 ng.

It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

The term, “DP-N”, as used herein, refers to the degree of polymerization, where N is the number of monomeric units (i.e., glucose or dextrose units) in the saccharide, thus DP-N reflects the composition of the carbohydrate. For example, DP1 is a monosaccharide and refers only to dextrose; DP2 is a disaccharide and refers only to maltose; DP1+2 is the total of monosaccharides and disaccharides; DP3−10 is the total of DP 3 to DP10; DP11+ is the total of saccharides DP11 and greater. Further, as used herein, DP3 refers only to maltotriose; DP4 refers only to maltotetraose; and DP5 refers only to maltopentaose.

DP-N is expressed as a weight percent of an individual saccharide on a total carbohydrate dry weight basis. Generally, as DP increases, the sweetness of a syrup decreases whereas its viscosity increases. The DP-N composition can be measured using high performance liquid chromatography (HPLC). HPLC is a technique in analytical chemistry used to separate, identify, and quantify each component in a mixture. HPLC relies on pumps to pass a pressurized liquid solvent containing the sample mixture through a column filled with a solid adsorbent material.

As an example, a sample of low-viscosity reduced-sugar syrup is diluted with deionized water to 0.5% to 5% DS, de-ashed with ion exchange resins (Dowex 66 and Dowex 88, Dow Chemical Co., Midland, Mich.), and filtered through a 0.45-micron filter before injection into the HPLC for DP carbohydrate analysis. DP separation can be accomplished using two BioRad Aminex HPX-42A, 300 mm×7.8 mm columns (BioRad, Hercules, Calif.) in series using water as the eluent at a flow rate of 0.20 ml/min at 65° C. Separated DP can then be quantitated with a refractive index detector.

The term, “oligosaccharide” as used herein, refers to a starch-derived product with a DP of from at least 3 to at most 14. For example, DP3-7 is an oligosaccharide, DP3-10 is an oligosaccharide; DP3-14 is an oligosaccharide; DP4-6 is an oligosaccharide. The term, “polysaccharide”, as used herein, refers to a starch-derived product with a DP of at least 11. For example, DP11+ is a polysaccharide.

### Wholegrain Syrup

As explained, syrups derived from whole grain are traditionally rich in sugar and are intended to impart sweetness or may be used as feedstock for brewing. Typical syrups do not perform well in applications that require low sugar or low hygoscopicity. A wholegrain syrup with minimal sweetness and a viscosity low enough to allow for easy handling is need in the field and is described herein.

The term, “syrup”, as used herein, refers to aqueous solutions of starch hydrolysates.

In all glucose polymers, from the native starch to glucose syrup, the molecular chain begins with a reducing sugar, containing a free aldehyde. As the starch is hydrolyzed, the molecules become shorter and more reducing sugars are present. The DE describes the degree of conversion of starch to dextrose. In particular, starch has a DE value close to 0 while glucose/dextrose has a DE value of 100. The DE value of maltodextrins varies between 3 and 20, while glucose syrups have a DE value more than 20. Pure maltopolymers range from low DE maltodextrins to high DE glucose syrups.

The term, “viscosity”, as used herein, refers to a resistance of a fluid to flow. The viscosity of a syrup is typically affected by temperature and solid concentration. Viscosity is expressed in terms of centipoise (cP) at a given temperature and a given % dry solids (DS). The viscosity of the syrup varies depending on the composition of the syrup, which is contingent on the amount of mashing of the whole grain seed and a length of time the mash is digested with an endoglycosidase.

In certain embodiments, at about 60% dry solids and at about 37° C. (or 98.6° F.), the viscosity of the syrup can be in the range from about 1000 to about 30,000 centipoise units (cps). In other embodiments, at about 78% dry solids and at about 37° C., the viscosity of the syrup can be in the range from about 5000 to about 30,000 cps or from about 10,000 to about 30,000 cps or from about 15,000 to about 30,000 cps or from about 20,000 to about 30,000 cps or about 25,000 to about 30,000 cps. In further embodiments, at about 78% dry solids and at about 37° C., the viscosity of the syrup can be in the range from about 5,000 to about 30,000 cps or from about 5,000 to about 25,000 cps or from about 5,000 to about 20,000 cps or from about 5,000 to about 15,000 cps or from about 5,000 to about 10,000 cps.

A Brookfield viscometer (model LVDV-E 115, Brookfield Engineering Inc., Middlesboro, Mass.) with a 12-mL small sample adapter was employed for the determination of the viscosity in examples described herein. A temperature of the small sample adapter was controlled using a circulation water bath. Spindle #S-25 was used while rotation speed was varied so that the percent torque fell between 25% to 75% during the viscosity measurements.

Moreover, a sweetness index (SI) is based on the proportion of individual sugar components present in a syrup composition. For example, the sweetness index can be calculated based on content and sweetness properties of individual carbohydrates by multiplying the sweetness coefficient of each sugar (glucose=1.00, maltose=0.3, fructose=2.30, and sucrose=1.35) with the concentration of that sugar as determined by HPLC analysis of each individual sugar. See, Lembe Samukclo Magwaza, et al., “Analytical methods for determination of sugars and sweetness of horticultural products-A review,” Scientia Horticulturac, 2015, Vol. 184, Pages 179-192, the content of which is incorporated by reference herein in its entirety. In this sweetness estimation approach, the contribution of each carbohydrate is calculated, based on maltose being 0.3 times as sweet as glucose. Hence, the level of sweetness can be calculated, for example, using the equation: SI=(1.00[glucose])+(0.30[maltose]). Alternatively, for a polydisperse mixture, an average or effective sweetness index can be estimated based on a comparison of the perceived sweetness vs. standard solutions of sucrose at various concentrations.

In certain embodiments, the sweetness index of the syrup can be about 0.01, about 0.01, about 0.02, about 0.03, about 0.04, about 0.05, about 0.06, about 0.07, about 0.08, about 0.09, about 0.1, about 0.11, about 0.12, about 0.13, about 0.14, or about 0.15.

As used herein, a whole grain seed or flour derived therefrom includes, but is not limited to, barley, rice, black rice, brown rice, wild rice, buckwheat, bulgur, corn, millet, oat, sorghum, spelt, triticale, rye and wheat. In certain embodiments, the whole grain can be sprouted. In other embodiments, the whole grain can be non-sprouted. In examples, the flour's nutritional profile comprises: protein 6 to 8.5 g/100 g, crude fat 1 to 3 g/100 g, crude fiber 0.5 to 5 g/100 g, mono and disaccharides <10 g/100 g, total carbohydrates 70 to 80 g/100 g. In other examples, the flour has at least 50% more surface area compared to equivalent standard flours as a result of having been partially dissolved and then spray dried. The wholegrain seed or flour is described further below.

In further examples, the whole grain component can be ground, preferably by dry milling. Such grinding may take place before or after the whole grain component is subjected to enzymatic and/or acid hydrolysis. In certain embodiments, the whole grain component can be heat treated to limit rancidity and microbial count. In certain embodiments, the whole grain seed may be sprouted.

In certain embodiments, the non-carbohydrate nutritional components of the syrup comprise fats, proteins, minerals and/or fibers.

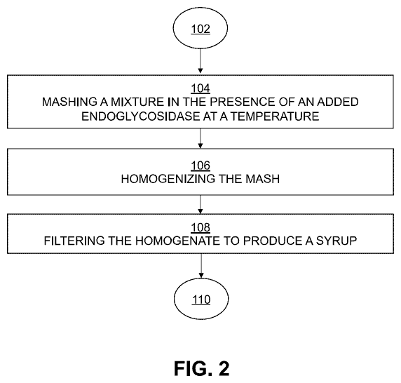

A method to create a low sugar and low viscosity syrup from whole grain seed or flour includes numerous process steps and is depicted in FIG. 2. The method includes the controlled hydrolysis of a whole grain seed or flour mash that has been cooked by jet or kettle cooking. By incubating the mash at about 37° C., one or more glycosidic hydrolases convert the starch into oligosaccharides. An acid conversion step is optional but is not preferred as acid doesn't hydrolyze as precisely as amylase.

The method of FIG. 2 begins at a process step 102. A process step 104 may follow the process step 102 and may include mashing a mixture in the presence of an added endoglycosidase at a given temperature. In examples, the mixture comprises a cooked whole grain cereal. In some examples, the mixture does not contain a refined starch and has not been fermented by a microorganism. In other examples, the temperature is approximately 70° C.

In some examples, the added endoglycosidase of the process step 104 of FIG. 2 comprises one or more glycosidic hydrolases. In examples, the endoglycosidase is an alpha amylase, such as a thermally stabile alpha amylase, a maltotetragnic alpha amylase, or an organic alpha amylase. In specific examples, the alpha amylase may be alpha-amylase BAN 480 L (Bacterial Amylase Novo, an 1,4-alpha-D-glucan glucano-hydrolase (EC 3.2.1.1)), produced by submerged fermentation of a selected strain of Bacillus amyloliquefaciens. The added endoglycosidase are substantially devoid of 1,4-alpha-D-Glucan and/or beta-amylase used in prior art references to create sweetening syrups. Such added endoglycosidase converts the starches into oligosaccharides. Optionally, other digestive enzymes may be deployed (peptidase, pectinases, pullanase, proteinases, and/or cellulases) to break down cell wall structures, enhance syrup extraction, and increase the levels of soluble fibers.

It should be appreciated that the intensity and/or duration of the mashing step (e.g., the process step 104) facilitates the enzymatic hydrolysis of the starch molecules, resulting in a more controlled hydrolysis to produce a syrup comprising oligosaccharides with a narrow molecular weight distribution. In certain embodiments, the enzymatic hydrolysis and mechanical treatment steps may be combined.

As described herein, the “refractive index” is a ratio of the speed of light in a medium relative to its speed in a vacuum. This change in speed from one medium to another is what causes light rays to bend because as light travels through another medium other than a vacuum, the atoms of that medium constantly absorb and reemit the particles of light, slowing down the speed light travels at. Thus, the denser the liquid, the slower the light will travel through it, and the higher its reading will be on the refractometer.

The refractive index can be calculated using the equation below.

$${n}_{D}^{t} = \frac{speed of light in vacuum}{speed of light in the medium}$$

Many different scales are available that convert refractive index into a unit of measure that is more meaningful, i.e., Brix, specific gravity, Plato, etc.

An exemplary method of measuring a refractive index or Brix of a composition is taught, for example, in U.S. Pat. No. 9,448,166, the content of which is incorporated by reference herein in its entirety.

As defined herein, “Brix” is a measurement in percentage by weight of sucrose in a pure water solution. Brix represents the physical/mathematical relationship between refractive index and the content of sucrose per weight in sucrose water solution.

“Density” refers to the quotient of the mass (m) and the volume (v) of a substance (mass density). As the density depends primarily on temperature, the latter must always be specified. The following equation may be used to calculate density: d=m/V. There is a direct relationship between density and Brix and the measured density can be easily converted directly into a weight percent sucrose content (e.g., a ° Brix).

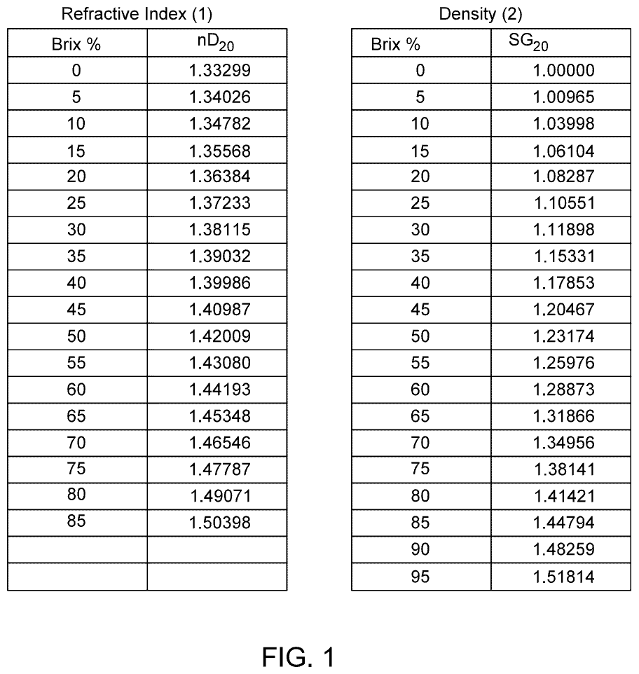

As a ray of light travels from one medium into another optically less dense one, the ray of light changes direction. With an increasing angle of incidence, the ray of light reaches a critical value at which no light escapes from the denser medium. If this critical angle is exceeded, total reflection occurs. The “refractive index” is calculated from this critical angle. The refractive index depends not only on the wavelength used to measure, but also on the temperature of the solution being measured. Such conversion between a refractive index and the Brix value at 20° C. and between the density and the Brix value at 20° C. is depicted in FIG. 1.

A process step 106 follows the process step 104 of FIG. 2 and includes homogenizing the mash of the process step 104. In some examples, the homogenate may be concentrated to a Brix value of at least about 60. Further, a process step 108 follows the process step 106 of FIG. 2 and includes filtering the mash to produce a syrup. A process step 110 follows the process step 108 to conclude the method of FIG. 2. It should be appreciated that there are several optional steps for the method of FIG. 2. For example, a clarification/filtration step is optional to reduce viscosity and produce a clarified syrup rich in soluble nutrients (minerals, fibers, phospholipids, and/or proteins). Additionally, a concentration step may be required to achieve a solid content above 60% that is conducive to a shelf-stable syrup.

The syrup described herein and formed from the method of FIG. 2 is a low sugar and low viscosity syrup created from whole grain seed or flour. By controlling the mashing and hydrolysis of the cooked whole grain seed or flour, the syrup can be obtained comprising a defined oligosaccharide content with a narrow molecular weight distribution (e.g., low in sugar and low DP 11+). The syrup can be used as a nutrient-rich bulking agent, binder, beverage thickener or as an encapsulating agent for active ingredients. In specific examples, the non-sweet syrup may be useful as a carrier.

In examples, the syrup created from the method of FIG. 2 has a sweetness index of less than about 0.25 and a viscosity not greater than about 30,000 cps units at about 78% dry solids and at about 37° C. In some examples, the syrup described herein has 90 to 100% of its solid components as water soluble solids. In other examples, the syrup described herein is created from a germinated whole grain seed and has 50 to 95% of its solid components as water-soluble solids. In other examples, the syrup comprises less than about 25% total monosaccharides and disaccharides on a dry weight basis.

Furthermore, in one embodiment, the syrup comprises greater than 55% oligosaccharides on a dry weight basis with a degree of polymerization of from about 3 to about 14. In other examples, the syrup comprises less than about 20% polysaccharides on a dry weight basis with a degree of polymerization of at least about 11 (DP 11+). Also, the syrup has about 1 to 15% of solids in the syrup comprise non-carbohydrate nutritional components.

In certain embodiments, dry products comprising the carbohydrate compositions according to the disclosure may be prepared by drying the syrups described herein to form a dry powdered product using methods well known in the art, for example by freeze-drying, spray drying, fluidized-bed drying, rotary drying, tunnel drying, tray or cabinet drying to produce a powdered dry product. Dry products typically have moisture levels of less than about 10% or less than about 5%.

In certain embodiments, the Brix value of the syrup is less than 60.

In certain embodiments, the Brix value of the syrup is about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49 or about 50. In preferred examples, the syrup as a Brix value in the range of about 10 to about 50.

### Wholegrain Flour

As described, the instant invention provides a sprouted wholegrain flour in which 50% to 95% of the composition is readily soluble in water. The flour retains its original nutritional profile of micronutrients and macronutrients and is suitable for replacing highly refined ingredients such as maltodextrin within prepared foods.

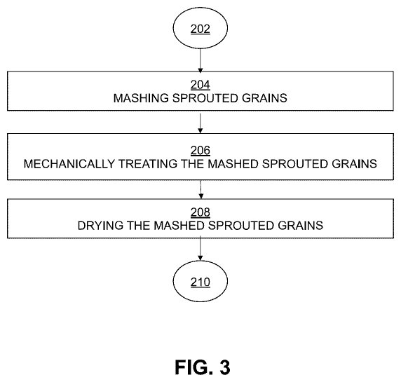

A method to produce the wholegrain flour is depicted in FIG. 3 and includes numerous process steps. The method of FIG. 3 requires a sprouted grain, which is required for providing naturally high levels of enzymes which can be deployed in a subsequent mashing step. The sprouted grain may be derived from a sprouted or germinated seed.

The method of FIG. 3 begins at a process step 202. A process step 204 follows the process step 202 that includes mashing the sprouted grains to activate endogenous enzymes of the sprouted grains and partially solubilize proteins and starches of the sprouted grains. In examples, the process step 204 increases a solubility of the sprouted grains to 50 to 95%, while maintaining the nutritional profile of flour.

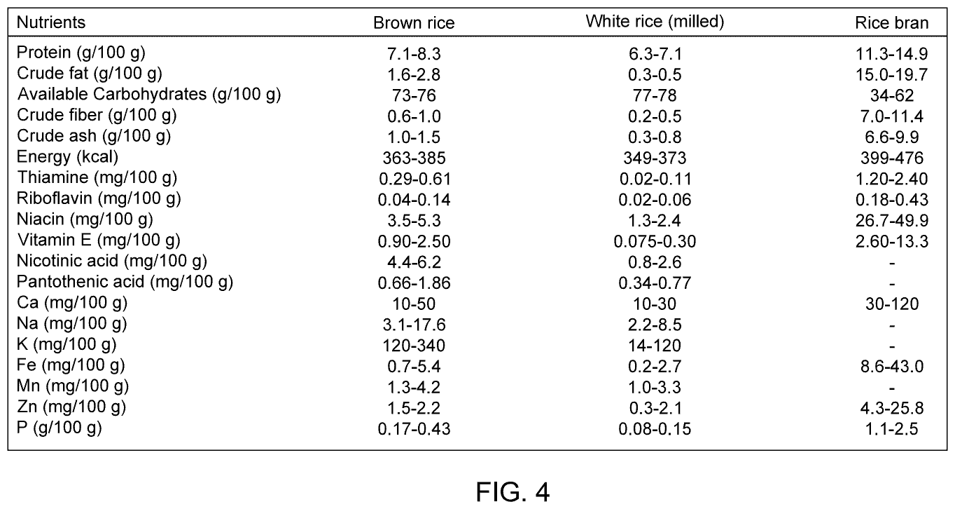

The process step 204 should avoid excessive sacchrification, which is the process of breaking down complex carbohydrates like corn into monosaccharide components, which would increase the sugar content and alter the nutritional profile beyond the range typical for the respective flour. A typical nutritional profile range for brown rice, milled rice, and rice bran is depicted in FIG. 4. See, Ahmed S. M. Saleh, et al., “Brown Rice Versus White Rice: Nutritional Quality, Potential Health Benefits, Development of Food Products, and Preservation Technologies,” Comprehensive Reviews in Food Science and Food Safety, 2019, Vol. 18, Pages 1070-1096, the contents of which are hereby incorporated in their entirety.

A process step 206 follows the process step 204 and includes mechanically treating the mashed sprouted grains. The mechanical treatment of the process step 206 reduces a particle size of remaining insoluble constituents such that the insoluble constituents can be more readily accommodated within food applications. A process step 208 follows the process step 206 and includes drying the mashed sprouted grains to create a wholegrain flour powder or granule. The process step 208 may include spray drying. A process step 210 may conclude the process steps of the method of FIG. 3. It should be appreciated that an optional filtration step may be included in the method of FIG. 3, but is not necessary.

The wholegrain flour powder produced from the method of FIG. 3 may be useful for plating of oily materials to render them into flowable powders. The wholegrain flour powder may also be useful as a nutrient-rich bulking agent in foods. In other examples, the wholegrain flour powder can be used as a binder for granola bars, to impart mouthfeel to thickened beverages, as a powder carrier for natural high potency sweeteners and as an encapsulating agent for active ingredients. In some examples, the wholegrain flour powder may be additionally useful for reducing powder cohesion and improving the powder flow properties of spice mixtures. Spice mixtures, such as ground pepper, can have oily constituents, which bridge between particles and make them difficult to disperse evenly onto food surfaces. Readily soluble wholegrain flours, in certain embodiments, can have high surface area which may be useful for absorbing excess oily constituents.

### EXAMPLES

### Example 1

A measure of 500 g of organic brown rice (365 brand) was combined with 700 g water ad cooked in a household rice cooker to create 895 g of cooked rice. An additional 600 g of cold water was added and the cooked rice was pulped in a Bellini model BMKM510CL cooker at speed 10 for 5 minutes. The preparation was heated to 70° C. and 700 uL of BAN 480L amylase enzyme preparation was added. Mashing continued at 70° C. for 5 minutes. The preparation was homogenized using a Silverson LMA-5 homogenizer at 10,000 rpm form 4 minutes and returned to mash at 70 C for an additional 10 minutes. The preparation was strained via a 75-micron mesh bag to produce 1156 of syrup and 79 g of wet rice solids. The rice syrup had a milky appearance and Brix value of 25 as read by an Atago AL-1 refractometer. The rice syrup was concentrated by boiling to 60 brix in the Bellini cooker using the SR setting. A preparation of the whole grain rice syrup was prepared at 25 brix. This was compared to a sugar solution at 2.5 Brix and found to be less sweet. The whole grain rice syrup had a sweetness index of <0.1.

### Example 2

A rice preparation created in Example 1 without the Silverson homogenization step. After filtration with a 75 micron nylon bag, 163 g of wet rice solid and 1326 g of rice syrup was produced. In this case the syrup was translucent and less opaque compared to the syrup of Example 1.

### Example 3

A measure of 800 g of malted sweet rice flour (ECKERT MALTING, Chico, CA) was combined with 1200 g of water at 70° C. The preparation was mashed for 90 minutes to achieve a low viscosity suitable for spraying. The preparation was homogenized using a Silverson LMA-5 homogenizer at 10,000 rpm form 4 minutes and returned to mash at 70° C. for an additional 10 minutes. The rice flour preparation has a milky appearance and Brix value of 34 as read by a Atago AL-1 refractometer. The flour preparation was transferred to an APV Anhydro LabS1 spray dryer and atomized using a centrifugal wheel and dried with inlet temperature 140° C. and outlet temperature of 100° C. Next, 600 g of spray dried rice flour was collected in an outlet cup.

### Example 4

Example 4 describes a solubility test. An aliquot of 4.91 g of the spray dried brown rice flour of Example 3 was combined with 45 g of water and shaken within a 50 mL centrifuge tube for 5 minutes. The preparation was then centrifuged at 4000 rpm for 10 minutes and decanted to collect a precipitate of the insoluble fraction. The precipitate was dried at 70° C. for eight hours to determine a dry weight of 0.6 g. The soluble fraction is calculated as 4.91-0.6 g/4.91 g or 87.8%.

### Example 5

100 g of the spray dried brown rice flour of Example 3 was combined with 150 g of Solo brand pure almond paste. The mixture was blended at high speed in a Bellini model Bmkm510cl blender. The paste was dispersed on to the rice flour powder creating a free-flowing powder mixture. The almond powder mixture was further combined with 65 g of cane sugar to create an almond beverage base.

### Example 6

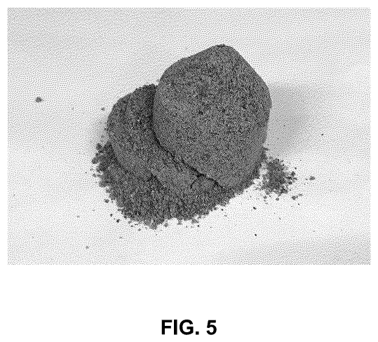

50 g of whole black pepper corn (McCormick brand, Baltimore, MD) was ground to a fine powder. A measure of 12.36 g of the ground pepper was placed into a 50 mL centrifuge tube and tapped by hand 100 time against a tabletop to pack the ground pepper into the tube. The tube was inverted and the plug of ground pepper slid out and largely held its shape. The plug of ground pepper is depicted in FIG. 5.

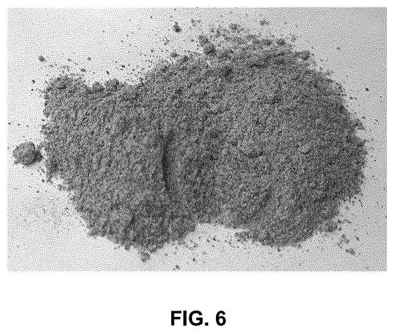

A separate aliquot of 12 g of ground pepper was combined with 6 gram of the spray dried rice flour of Example 3. The powder mixture was stirred with a whisk for 2 minutes. Approximately 12.96 g of the black pepper rice flour mixture was placed into a centrifuge tube and tapped 100 times. The tube was inverted and the mixture slide out without holding the shape of the plug is depicted in FIG. 6.

Aspects of the present invention are described herein with reference to methods and systems according to embodiments of the invention. The block diagrams in the Figures illustrate the architecture, functionality, and operation of possible implementations of methods according to various embodiments of the present invention. In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others or ordinary skill in the art to understand the embodiments disclosed herein.

Any patent, patent application, publication, or other disclosure material identified in the specification is hereby incorporated by reference herein in its entirety. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict between the present explicit disclosure and a document incorporated by reference, the present explicit disclosure shall be the operative disclosure.

What is claimed is:

1. A syrup, derived from whole grain or a whole grain flour, the syrup having a composition comprising:
less than 25% of monosaccharides and disaccharides in a dry weight basis;
greater than 55% of oligosaccharides in the dry weight basis;
less than 20% of polysaccharides in the dry weight basis; and
non-starch derived nutritional components from a grain and present in a range of approximately 1% to approximately 15%, wherein the whole grain or the whole grain flour is derived from barley, rice, black rice, brown rice, wild rice, buckwheat, bulgur, corn, millet, oat, sorghum, spelt, triticale, rye or wheat, wherein a Brix value of the syrup is less than 60.

2. The syrup derived from the whole grain or the whole grain flour of claim 1,
wherein the syrup comprises 90% to 100% of its solid components as water soluble solids.

3. The syrup derived from the whole grain or the whole grain flour of claim 1,
wherein the monosaccharides and disaccharides comprise a sweetness index below approximately 0.15.

4. The syrup derived from the whole grain or the whole grain flour of claim 1,
wherein the polysaccharides have a degree of polymerization of at least 11.

5. The syrup derived from the whole grain or the whole grain flour of claim 1,
wherein the non-starch derived nutritional components are selected from the group consisting of: proteins, fats, minerals, cellulose, and soluble fibers.

6. The syrup derived from the whole grain or the whole grain flour of claim 1, wherein the whole grain or the whole grain flour does not contain a refined starch and has not been fermented by a microorganism.

7. The syrup derived from the whole grain or the whole grain flour of claim 1,
wherein the syrup is used as a food binder, an encapsulating agent, a humectant, a mouthfeel enhancer, a dispersant, a solvent, a carrier, an adhesive, and a metabolic energy source.

8. The syrup derived from the whole grain or the whole grain flour of claim 1,
wherein the syrup is derived from a sprouted wholegrain source without use of acid hydrolysis and without use of exogenous enzymes.

9. The syrup derived from the whole grain or the whole grain flour of claim 8, wherein no exogenous amylase is added to the sprouted wholegrain source.
